(19) United States
(12) Plant Patent Application Publication
(10) Pub. No.: US 2002/0002728 P1
(43) Pub. Date: Jan. 3, 2002

(54) Hosta plant 'Morning Light'

(76) Inventors: Gert van Eijk Bos, Woerden (NL); Dirk van Erven, Ryswajk (NL)

Correspondence Address: Gert van Eijk-Bos, Vitro Westland B.V., Haantje 17-C, Rijswijk, 2288CV, NL

(21) Appl. No.: 09/921,934

(22) Filed: Aug. 6, 2001

(30) Foreign Application Priority Data
Aug. 16, 1999 (EP) 19991165

(51) Int. Cl. A01H 5/00

(52) U.S. Cl. PLT/353

(57) ABSTRACT

A new and distinct Hosta plant named ‘Morning Light’, characterized by very thick leaves, deep green margin, and a creamy yellow to creamy white center, that displays a unique upright habit and light lavender flowers held above the foliage on thick cream-colored scapes.

### BACKGROUND OF THE INVENTION

The present invention relates to the new and distinct cultivar of Hosta, botanically known as Hosta hybrid (Tratt.), and hereinafter referred to as the cultivar ‘Morning Light’.

The new plant was discovered by the inventors, Gert van Eijk-Bos and Dirk van Erven, as a non-induced, naturally occurring whole plant sport mutation of Hosta ‘Loyalist’ (not patented) at a nursery in Rijswijk, The Netherlands. Asexual propagation of the plant at the same nursery in Rijswijk, The Netherlands by tissue culture and division has shown that the unique and distinct characteristics of this new plant are stable and reproduce true to type in successive generations of asexual propagation.

The cultivar name, ‘Morning Light’, has been registered with the American Hosta Society, which is the International Cultivar Registration Authority for the Genus Hosta.

### SUMMARY OF THE INVENTION

Hosta ‘Morning Light’ is unique from its parent sport, Hosta ‘Loyalist’ (not patented) and all other hosta cultivars, in several traits. Hosta ‘Loyalist’ is a tissue culture derived sport of Hosta ‘Patriot’ (not patented), and ‘Patriot’, in turn, is a sport of Hosta ‘Francee’ (not patented). Hosta ‘Francee’ and ‘Patriot’ both have creamy white margins with the former having a thinner margin, and the latter, wider margins. Hosta ‘Morning Light’ has a reverse type variegation pattern, with a more yellowish pigmented center. Hosta ‘Loyalist’ (also a reverse type variegation pattern) has a paler white center earlier in the season. Both cultivars lighten in the center with more heat or light, but ‘Morning Light’ stays more pigmented at all times during the growing season. ‘Morning Light’ is also a more rapid grower, and reaches saleable size in a 3 inch×4½ inch deep pot in about 5½ months, that is about two weeks faster than ‘Loyalist’. Hosta ‘Morning Light’ has very thick, rigid leaves giving the whole plant an upright form and habit. The leaf blades and petioles have a wide dark green margin and yellowish cream to white centers. ‘Morning Light’ flowers in mid summer with light lavender flowers, on scapes well above the foliage.

### BRIEF DESCRIPTION OF THE DRAWINGS

The photographs of the new invention demonstrate the overall appearance of the plant including the unique traits. The colors are as accurate as reasonably possible with color reproductions.

FIG. 1 shows a young leaf in the early part of the season with the intricate leaf colors.

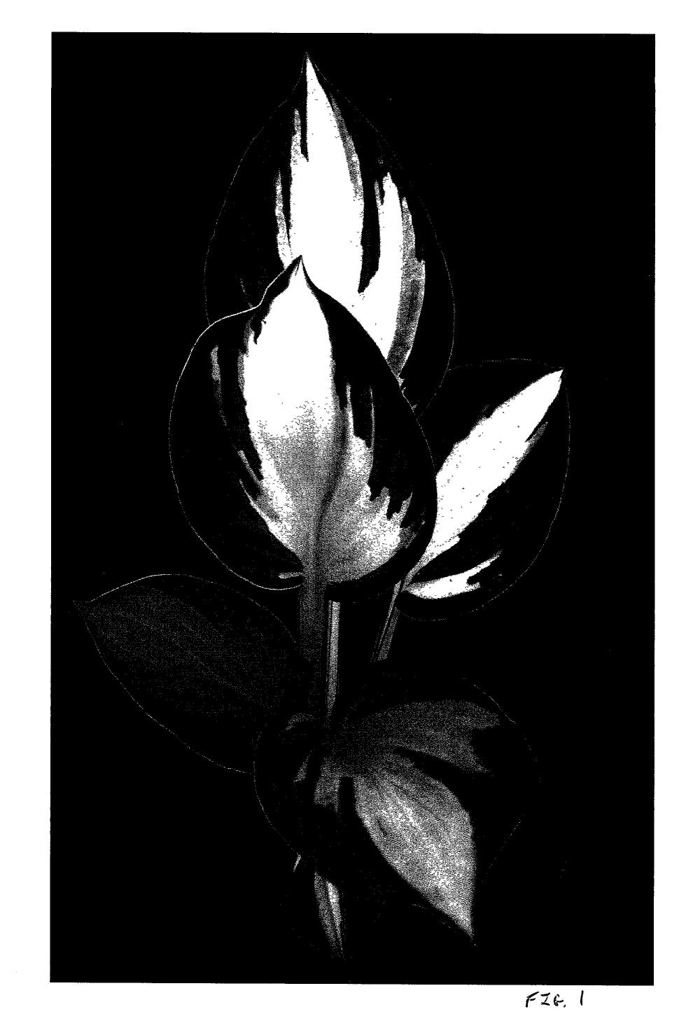

FIG. 2 shows a close-up of a more mature leaf later in the season with the multiple leaf colors.

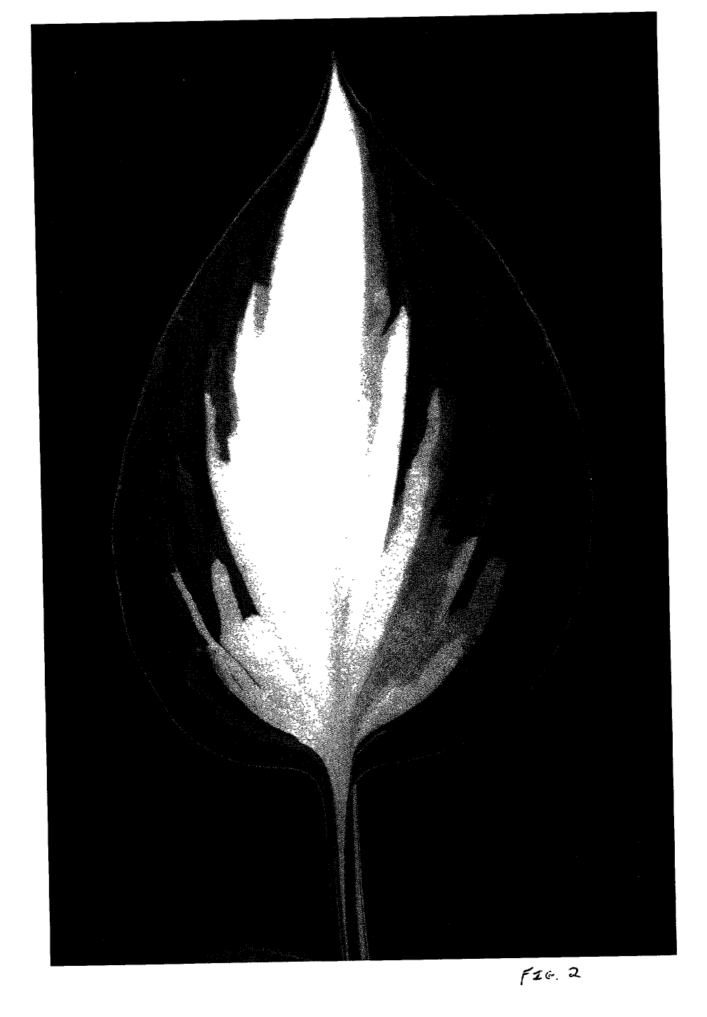

FIG. 3 shows the scape, flower and buds of the plant.

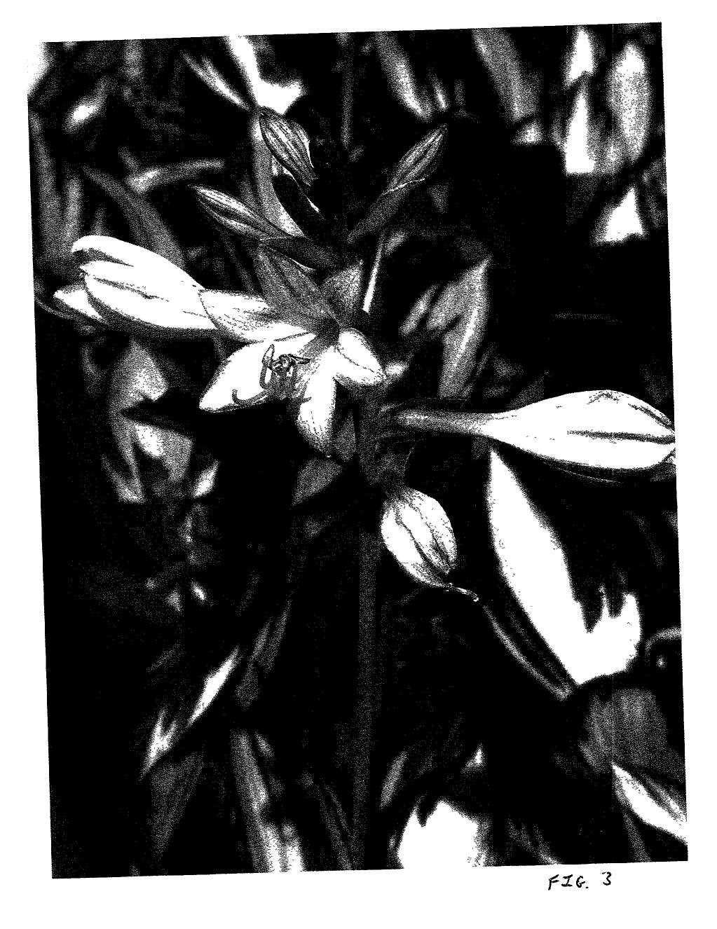

### DETAILED BOTANICAL DESCRIPTION

The following descriptions and color references are based on The Royal Horticultural Society Colour Chart except where common dictionary terms are used. The new plant, Hosta ‘Morning Light’, has not been observed under all possible environments. The phenotype may vary slightly with different environmental conditions, such as temperature, light, fertility and moisture, but without any change in the genotype. The following observations and size descriptions are of a two year old plant in a one gallon container grown in Zeeland, Mich., USA under 50% shade on cloudless days, day temperatures of 18 to 28 degrees C., and night temperatures of 15 to 20 degrees C.

Botanical classification: Hosta hybrid cultivar ‘Morning Light’.

Parentage: Naturally occurring sport of Hosta ‘Loyalist’ (not patented).

Propagation:

Method.—By tissue culture and division.

Time to initiate roots from both division and tissue culture.—About four weeks.

Rooting habit.—Normal, fleshy, freely branching.

Plant description:

Plant shape and habit.—Symmetrical, upright and erect through flowering period. More horizontal in older plants.

Crop time.—Under normal growing conditions about 22 weeks to finish in a one liter container. Plant vigor is good to high.

Plant size.—At flowering is 25 to 33 cm tall and 23 to 28 cm wide.

Foliage description:

Shape and size.—Ovate to broadly ovate leaf blades have an acute apex and cordate base. The leaf blades are 13 to 16 cm long and 8 to 10 cm wide. The leaf blade margin is entire and the variegation pattern on the edge varies in different regions of the leaf from 10 to 25 mm wide.

Color.—The adaxial surface is a deep green closest to RHS 141 A on the margin of young emerging leaves. Later in the growing season, the margin is between RHS 139 A and RHS 136 A. As leaves are emerging, the inside, or center of the leaf is yellow-green, more green than RHS 151 C. The center of the leaves progresses to yellow RHS 11 D to RHS 13 D later in the growing season. The abaxial margin surface is RHS 137 B on young leaves, and RHS 137 A on leaves later in the season. The abaxial center color starts RHS 154 D and lightens to RHS 4 D with light and heat. There are also three or more intermediate color patterns between the center and the margin. The most prominent color band is yellow-green RHS 144 B on newly emerged leaves, and between RHS 143 B and RHS 143 C on older leaves. Other more yellow, lighter, or more grayed bands are also sometimes present in thinner and shorter stripes. The main intermediate band visible on the abaxial side is RHS 144 C on young leaves and about RHS 145 C on leaves later in the season. The thinner and shorter bands on the abaxial side are comparable, but lighter than those on the adaxial surface. Petioles are 18 to 22 cm long and 6 to 9 mm wide. The center color starts the same as the leaf center and lightens to RHS 11 D, but varies like the leaves depending on how much light the petiole receives. The edge of the petiole is green RHS 136 A on both surfaces.

Flower description:

Buds.—Two days prior to opening the buds are violet RHS 85 D and RHS 85 A, 6 to 7 cm long, up to 1.5 cm wide, and when opened the flowers have tepals of Red purple RHS 69D and violet stripes of RHS 84 B.

Flowers.—15 to 21, funnelform, 4 to 6 cm wide and 6 to 8 cm long, (distal flowers being smaller) subtended by a bract 2 cm long and 5 to 7 mm wide with the same colors and pattern as the leaves, persists for a normal period, up to two days, and the scapes remain effective from mid July to late August.

Peduncle.—Erect to 48 cm tall and 0.8 cm in diameter, cream color RHS 11 D with tints of green closer to the base, and a reddish purple stippling RHS 60 B becoming much heavier at the apical one fifth.

Pedicel.—Approximately 1 cm long, 3 mm wide, RHS********.

Tepal.—Six fused at the base and acute apex, approximately 7 cm long and 1.2 cm wide, arranged in two layers of three, the inner three with clear 1 to 2 mm margin, overall color, tepals of Red purple RHS 69D and violet stripes of RHS 84 B .

Gynoecium.—Style— 5 to 6 cm long, 1 mm diameter, curled at distal end; Stigma to 3 mm .

Androecium.—Filaments— six, white, approximately 1 mm in diameter and to 5 cm long; Anthers — 5 to 6 mm long, 1.5 mm wide, about RHS 83 A around margin of abaxial side, white in center, pollen is yellow-orange RHS 17 B. Pods are variable in color similar to the peduncles and pedicels with the amount of reddish pigment determined by amount of light. Seeds are single winged dark brown drupe about RHS 200 A to RHS N200 A, 12 to 15 mm long and 3 to 4 mm wide.

Disease resistance.—The plant is more resistant to melting-out than most other light-centered varieties, a condition where the light colored leaf center becomes necrotic. It grows best with plenty of moisture, but is able to tolerate some drought. Hardiness and other disease resistance are typical of other hostas.

We claim:

1. A new and distinct cultivar of ornamental Hosta plant named ‘Morning Light’ as herein described and illustrated, suitable as a potted plant, for the garden, and for fresh-cut arrangements.
